(19) United States
(12) Patent Application Publication
(10) Pub. No.: US 2024/0342467 A1
(43) Pub. Date: Oct. 17, 2024

(54) HEATING PAD WITHOUT ELECTRIC FIELD RADIATION

(71) Applicant: Ying Wang, Suzhou (CN)

(72) Inventor: Ying Wang, Suzhou (CN)

(21) Appl. No.: 18/298,919

(22) Filed: Apr. 11, 2023

(51) Int. Cl. A61N 1/16; A61F 7/02

(52) U.S. Cl. CPC A61N 1/16; A61F 7/02; A61F 2007/0071; A61F 2007/0244

(57) ABSTRACT

A heating pad without electric field radiation is disclosed. The heating pad without electric field radiation includes a heating wire, a shielding layer, a cotton layer and flannel. The shielding layer is disposed on the heating wire, and the shielding layer is symmetrically provided with two groups. The cotton layer is disposed on the shield layer, the cotton layer is symmetrically provided with two groups. The flannel is disposed on the cotton layer, and the flannel is symmetrically provided with two groups. The present disclosure relates to the field of heating pads, and specifically to a heating pad without electric field radiation.

### TECHNICAL FIELD

The present disclosure relates to the field of heating pads, and specifically to a heating pad without Electric field radiation.

### BACKGROUND

Heating wires through the current will produce electric fields, generally depending on the number of coils, trough the size of the current, it will produce different degrees of electric fields. We live in the range of various electric fields radiation, with the pursuit of quality of life, more people are beginning to pay attention to the impact of electric fields on the human body.

After testing, it was concluded that by adding a shielding layer, the electric field radiation of a product with a heating wire may be reduced to zero radiation. However, the shielding layer may have a slight reduction in shielding effect after several machine washings, mainly in whether a coating on the shielding material will come off after so many machine washings.

The existing heating pads have no precedent for elimination of electric field radiation generated by the heating pads. A radiation of the existing heating pads available on the market is basically E-FIELD 100 V/M to 300 V/M, H-FIELD Peak value is greater than 20 mG

Based on a Faraday Cage effect, as an out layer of insulation of the product or wire is wrapped with wire mesh, or wrapped/coated with conductive composite material, which may effectively shield electric fields, and such shield layer structure is mostly used in communication cables.

The present disclosure can effectively shield and eliminate electric field radiation generated by heating pad products.

### SUMMARY

Based on above, to overcome the defects of the existing technologies, the present disclosure provides a heating pad without electric field radiation.

In some embodiments, the heating pad without electric field radiation includes a heating wire, a shielding layer, a cotton layer and flannel. The shielding layer is disposed on the heating wire, and the shielding layer is symmetrically provided with two groups, on upper and lower layers of the heating wire, thereby the heating wire as a whole is wrapped in the shielding layer. The shielding layer is then formed similar to a “Faraday Cage”, which can shield electric field radiation. The cotton layer is disposed on the shield layer, the cotton layer is symmetrically provided with two groups; and the flannel is disposed on the cotton layer, and the flannel is symmetrically provided with two groups.

In some embodiments, the shielding layer is a shield fabric, anti-magnetic cloth or RFID shielding fabric.

Preferably, the shield layer may also wrap around the heating wire, such structure similar to a structure of a shield layer applied in communication cables (a main function is to shield interference). Moreover, the shield layer may also be composited or sewn in an inner layer of a jacket of the heating pad, the jacket of the heating pad is a removable jacket, making it easy to clean the jacket of the heating pad or replace the jacket of the heating pad. The ordinary heating wire as a whole is wrapped in a way similar to a “Faraday Cage”, thus the electric field radiation generated when the current passes through the heating wire can be effectively shielded.

The present disclosure has following advantages by using above structure. The radiation of the existing heating pads available on the market is basically E-FIELD 100 V/M to 300 V/M. By adding the shielding layer, the electric field radiation of the heating pad being sold on the market can be reduced to zero radiation.

With an advancement of technologies, cost of shielding materials has been reduced, which makes it possible to apply it in bulk to everyday products.

The shielding fabric with thermal conductivity properties, which allows for more uniform coverage of the heat generated by the heating wire, thereby enhancing the overall heating area of the heating pad to be more evenly heated.

### BRIEF DESCRIPTION OF THE DRAWINGS

FIG. 1 illustrates a cross-sectional view of a heating pad without electric field radiation according to some embodiments of the present disclosure.

FIG. 2 illustrates a three-dimensional exploded view of a heating pad without electric field radiation according to some embodiments of the present disclosure.

Herein, 1 is heating wire, 2 is shielding layer, 3 is cotton layer, 4 is flannel.

### DETAILED DESCRIPTION OF THE EMBODIMENTS

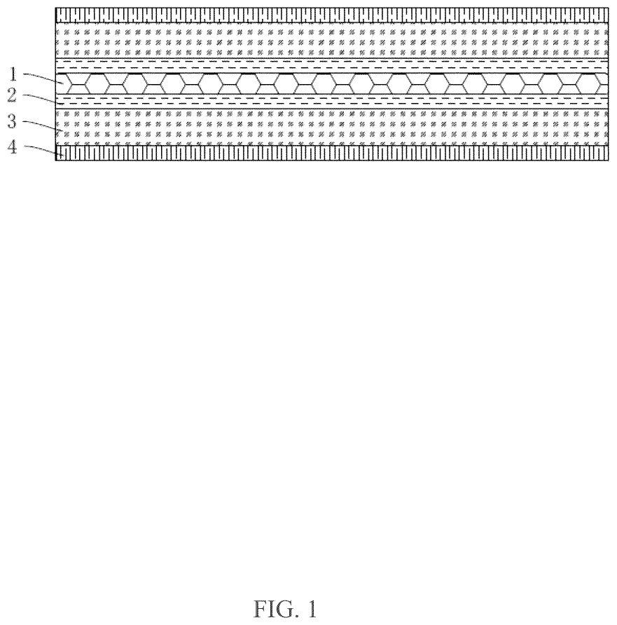

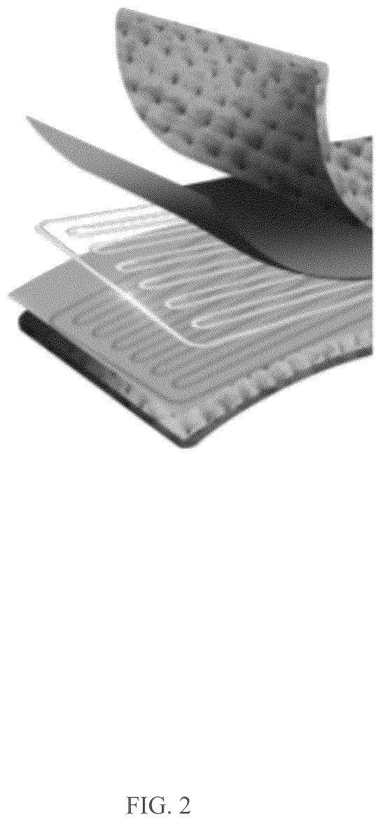

Referring to FIG. 1 and FIG. 2, which show a heating pad without electric field radiation according to some embodiments of the present disclosure. The heating pad without electric field radiation includes a heating wire 1, a shielding layer 2, a cotton layer 3 and flannel 4. Herein, the shielding layer 2 is disposed on the heating wire 1, and the shielding layer 2 is symmetrically provided with two groups. The cotton layer 3 is disposed on the shield layer 2, and the cotton layer 3 is symmetrically provided with two groups. The flannel 4 is disposed on the cotton layer 3, and the flannel 4 is symmetrically provided with two groups. In some embodiments, the shielding layer 2 is a shield fabric, anti-magnetic cloth or RFID shielding fabric.

When specific use, as the impact of electric field radiation on the human body is being paid more and more attention to, and the heating pad close contact with the human body (or use all night) for a long time 100 V/M to 300 V/M of electric field radiation on the human body shall not be ignored. So the electric field radiation of the heating pad can be shielded, which is the main purpose of the present disclosure.

It should be understood that although this specification is described according to embodiments, no each embodiment contains only one separate technical solution, the specification is described in this manner only for clarity, and those skilled in the art should consider the specification as a whole, and technical solutions in each embodiment may also be suitably combined to form other embodiments as may be understood by those skilled in the art.

I claim:

1. A heating pad without electric field radiation, comprising a heating wire, a shielding layer, a cotton layer and flannel; wherein the shielding layer is disposed on the heating wire, and the shielding layer is symmetrically provided with two groups;
the cotton layer is disposed on the shield layer, the cotton layer is symmetrically provided with two groups; and the flannel is disposed on the cotton layer, and the flannel is symmetrically provided with two groups.

2. The heating pad without electric field radiation according to claim 1, wherein the shielding layer is a shield fabric, anti-magnetic cloth or RFID shielding fabric.
